(19) United States
(12) Patent Application Publication
(10) Pub. No.: US 2016/0108437 A1
(43) Pub. Date: Apr. 21, 2016

(54) RAW SUGAR AND ETHANOL PRODUCTION METHOD USING SELECTIVE FERMENTATION

(71) Applicants: ASAHI GROUP HOLDINGS, LTD., Tokyo (JP); SHINKO SUGAR MILL CO., LTD., Osaka (JP)

(72) Inventors: Satoshi OHARA, Moriya-shi, Ibaraki (JP); Kazutoshi KITAI, Kashiwa-shi, Chiba (JP); Hidenori HIDAKA, Kumage-gun, Kagoshima (JP); Megumi SHIOURA, Nishinoomote-shi, Kagoshima (JP); Yosuke HAMADA, Kumage-gun, Kagoshima (JP)

(73) Assignees: SHINKO SUGAR MILL CO., LTD., Osaka-shi, Osaka (JP); ASAHI GROUP HOLDINGS, LTD., Tokyo (JP)

(21) Appl. No.: 14/787,553

(22) PCT Filed: May 14, 2014
(86) PCT No.: PCT/JP2014/062861

§ 371 Date: Oct. 28, 2015

(30) Foreign Application Priority Data
May 28, 2013 (JP) 2013-112078

(51) Int. Cl. C12P 7/06; C13B 20/16; C13B 30/02

(52) U.S. Cl. CPC C12P 7/06; C13B 30/02; C13B 20/165

(57) ABSTRACT

A method for producing raw sugar and ethanol which includes the steps of: heating and clarifying a plant origin sugar juice; concentrating the clear sugar juice so that the Brix value of the clear sugar juice is 15 to 50%; cooling the syrup to a fermentation temperature; fermenting the syrup, thereby selectively converting the saccharide components other than sucrose in the syrup into ethanol; and concentrating the fermented solution.

### TECHNICAL FIELD

The present invention relates to a method for producing raw sugar and ethanol, and more specifically, relates to a method for producing raw sugar and ethanol wherein sugar juice originated from a plant is fermented.

### BACKGROUND ART

Ethanol for fuel originated from a plant is expected to be a liquid fuel alternative to gasoline to prevent increase in carbon dioxide gas, and a method for producing ethanol by fermenting sugar juice originated from a plant with microorganisms has been conventionally investigated. However, there is a problem that consumption of sugar juice originated from a plant as a raw material for production of ethanol puts pressure on production of raw sugar, which is food.

As a method for solving this problem, Patent Document 1 describes a method for producing raw sugar and ethanol which can cover almost all of the energy consumed in a production process of raw sugar and ethanol by the energy obtained by burning a squeezed residue from sugar cane without causing decrease in the amount of raw sugar.

In addition, Patent Document 2 describes a method wherein a plant-origin sugar juice is first fermented with a yeast having no sucrose degrading enzyme, the fermented solution is clarified by heating and filtrating, the clarified sugar juice is concentrated to separate ethanol contained in the fermented sugar juice, sucrose is crystallized to produce raw sugar and ethanol, in order to further improve production efficiency of raw sugar and ethanol. The process is characterized in that conventional raw sugar producing steps are utilized, that is, the concentration steps, which have been used for evaporating aqueous components in the sugar juice, are utilized to evaporate ethanol at the same time.

A plant-origin sugar juice, for example, a sugarcane squeezed juice has a sugar concentration and a temperature which are suitable for conducting ethanol fermentation by using yeast. A plant-origin sugar juice, for example, a sugarcane squeezed juice, generally, heated by first, followed by conducting sterilization of microorganisms originated from raw materials and sedimentation of proteins in the sugar juice, through a clarifying step in which additives such as lime and a coagulation sedimentation agent are incorporated to separate the foreign substances by sedimentation, and then, is used for producing sugar or ethanol. As a result, the sugar juice after the clarifying step has a high temperature which is not suited for ethanol fermentation. For this reason, the process of patent document 2 is characterized in that the fermentation step is conducted with a sugar juice before subjected to the clarifying step.

The process of patent document 2, however, ferments an unsterilized plant-origin sugar juice before heating. Therefore, when the fermentation period is prolonged with the sugar juice containing a large amount of, for example, invert sugar, the amount of sucrose degraded by incorporation of microorganisms other than yeast during fermentation of sugar juice is large, and it is difficult to increase the yield of raw sugar. That kind of microorganisms also convert the degraded saccharide components into other substances such as lactic acid, acetic acid or the like. Therefore, there is a limit on increasing the yield of ethanol. In addition, since the plant-origin sugar juice generally contains a large amount of foreign substances and microorganisms, it is difficult to repeatedly utilize yeast, and an efficient fermentation method wherein especially a flocculent yeast is always present in a fermenter to continuously carry out fermentation without separation of the yeast is difficult. Additionally, there is a problem that, when the heated fermented solution is stood still in a precipitation tank in the clarifying step after fermentation, since the precipitation tank is generally that of air open system, a part of heated alcohol is evaporated and the final amount of recovery of ethanol decreases.

The specification of PCT/JP2013/07459 describes a method for producing raw sugar and ethanol by heating and clarifying a sugar juice squeezed from a plant, then fermenting the obtained clear sugar juice, and thereafter concentrating the fermented solution. By means of clarifying the sugar juice before ethanol fermentation, effects of prevention of contamination with microorganisms, improvement in the yield of raw sugar and ethanol and the like can be obtained. Thus, the above-mentioned problems can be solved by this method.

However, taking necessity of implementing the method on an industrial scale as a business into consideration, it is desired to more improve energy efficiency of the above-mentioned method for producing raw sugar and ethanol.

Patent document 3 describes that glucose in an aqueous solution of substrates which comprise sucrose and fructose polymer is selectively subjected to ethanol fermentation by using a yeast capable of fermenting glucose to alcohol but incapable of hydrolyzing fructose polymer or sucrose. The substrates which comprise sucrose and fructose polymer are prepared by applying fructosyltransferase and glucose isomerase at the same time to a sucrose containing substrate. As the sucrose containing substrate, molasses and the like are exemplified.

The invention of patent document 3 aims at producing a sweet syrup having high fructose content by using molasses and the like as raw materials. The molasses is residue left after sugar is crystalized and recovered from sugar juice, that is, residue obtained from conventional process for producing raw sugar. The invention of patent document 3 is, however, not the process utilizing the conventional raw sugar producing steps as patent document 2 is, and the objective product is also different. The syrup containing a large amount of fructose is low in sucrose content, that is, not only glucose but also sugar is consumed.

The present invention aims at increasing yield of raw sugar which is sucrose crystal, and relates to the art for increasing the purity of sucrose of a sugar juice, that is, the content rate of sucrose occupied in the whole soluble solid, by selectively fermenting glucose and fructose, to improve crystal recovery efficiency of sugar. Therefore, the invention of patent document 3 is different in objective from the present invention.

### BACKGROUND ART DOCUMENTS

### Patent Documents

[Patent Document 1] Japanese Patent Laid-open Publication No. 2004-321174

[Patent Document 2] Japanese Patent No. 4883511

[Patent Document 3] U.S. Pat. No. 4,335,207

### SUMMARY OF THE INVENTION

### Problems to be Solved by the Invention

The present invention solves the above-mentioned conventional problems, and an object thereof is to provide a method for producing raw sugar and ethanol by the use of conventional sugar producing steps, which does not degrade sucrose during fermentation, which increases recovery amount of raw sugar, and at the same time, which increases recovery amount of ethanol.

### Means for Solving the Problems

The present invention provides a method for producing raw sugar and ethanol comprising the steps of

heating and clarifying a plant-origin sugar juice;

concentrating the clear sugar juice so that Brix value of the clear sugar juice is 15 to 50%;

cooling the concentrated clear sugar juice to a fermentation temperature;

fermenting the concentrated clear sugar juice, thereby selectively converting the saccharide components other than sucrose in the concentrated clear sugar juice into ethanol; and

concentrating the fermented solution.

In addition, the present invention provides a method for producing raw sugar and ethanol comprising the steps of:

heating and clarifying a plant-origin sugar juice;

introducing the clear sugar juice into a multiple-effect evaporator tube;

concentrating the clear sugar juice by letting the clear sugar juice pass through an evaporator tube placed in the first position of the multiple-effect evaporator tube and thereafter drawing the clear sugar juice before introducing the clear sugar juice into an evaporator tube placed in the last position of the multiple-effect evaporator tube;

cooling the concentrated clear sugar juice to a fermentation temperature;

fermenting the concentrated clear sugar juice, thereby selectively converting the saccharide components other than sucrose in the concentrated clear sugar juice into ethanol;

heating the fermented solution to a concentration temperature; and

concentrating the fermented solution by letting the fermented solution pass through an evaporator tube placed in the next position of the evaporator tube from which the concentrated clear sugar juice is drawn.

In one embodiment, Brix value of the clear sugar juice is adjusted to 15 to 40% by letting the clear sugar juice pass through the evaporator tube placed in the first position of the multiple-effect evaporator tube and thereafter drawing the clear sugar juice before introducing the clear sugar juice into the evaporator tube placed in the last position of the multiple-effect evaporator tube.

In one embodiment, the fermentation is carried out using a sucrose unassimilating yeast.

In one embodiment, the fermentation is carried out using a yeast having no sucrose degrading enzyme.

In one embodiment, the fermentation is carried out in the presence of a sucrose degrading enzyme inhibitor.

In one embodiment, the plant is at least one kind selected from the group consisting of sugar cane, sugar beet, sugar palm, sugar maple and sorghum.

### Effects of the Invention

By means of the method of the present invention, since fermentation is carried out using heated and clarified sugar juice, even when the fermentation period is prolonged in the sugar juice containing a large amount of invert sugar, sucrose is hardly degraded during fermentation of the sugar juice; yield of the raw sugar is large; and at the same time, yield of the ethanol is large. In addition, since the sugar juice to be subjected to fermentation has been subjected to inactivation of microorganisms by heating and to clarification by removing foreign substances, it hardly occurs that the yeast is contaminated with incorporated microorganisms or with foreign substances, and recovery and reuse of the yeast can be easily carried out. Furthermore, in the case where the clear solution is utilized, microorganisms or foreign substances are not accumulated in a fermenter, since a yeast having a flocculent property becomes available, a yeast separator becomes unnecessary, thereby, shortening of process time becomes possible. Additionally, since concentration is directly carried out without using a precipitation tank after fermentation, loss of ethanol due to evaporation in the precipitation tank can also be avoided.

Furthermore, the method of the present invention is excellent in heat utilization efficiency, and also excellent in production efficiency of ethanol. Here, the production efficiency of ethanol means a production amount of ethanol per time or a production amount of ethanol per equipment volume. In addition, the method of the present invention enables downsizing of fermentation equipment, reduction in installation cost and the like.

### BRIEF DESCRIPTION OF THE DRAWINGS

FIG. 1 A flow diagram of the process used in Reference Example 1.

FIG. 2 A diagram showing material balance of the process of Reference Example 1.

FIG. 3 A diagram showing material balance of the process of Comparative Example 1.

FIG. 4 A flow diagram of the process which is one example of the present invention.

FIG. 5 A diagram showing material balance of the process of Example 1.

FIG. 5 A diagram showing material balance of the process of Example 2.

FIG. 5 A diagram showing material balance of the process of Example 3.

FIG. 5 A diagram showing material balance of the process of Example 4.

### MODE FOR CARRYING OUT THE INVENTION

In the method of the present invention, the plants to be used as a raw material of the sugar juice are plants which can accumulate saccharide components. Among them, a so-called crop as a raw material of crystal sugar is preferable. Specific examples of the crop as a raw material of crystal sugar include sugar cane, sugar beet, sugar palm, sugar maple, sorghum and the like. Preferable plants are sugar cane and sugar beet, and an especially preferable plant is sugar cane. This is because these plants accumulate a large amount of saccharide components. In addition, there are sugar producing factories using these plants as raw materials, and it is easy to put the present invention into practice.

The plant-origin sugar juice refers to a solution obtained by extracting saccharide components from a plant. The plant-origin sugar juice generally includes squeezed juice obtained by compressing a part in which the saccharide components of the plant is accumulated, broth prepared by decocting the part in which the saccharide components of the plant is accumulated, and the like.

Usually, the plant is cut and/or crushed into an appropriate size before being compressed or decocted. A means for squeezing juice such as a roll mill or the like may be used for compression of a plant. In addition, upon decocting a plant, the plant may be put into warm water for heating, or decoction means such as a diffuser may be employed. The temperature of the warm water as compressing and decocting period may be appropriately determined taking extraction efficiency of saccharide components into consideration. The temperature is usually 30° C. to 40° C.

In order to inactivate sucrose degrading enzymes, and to let proteins in the sugar juice modified, precipitated and sedimented, heating of the sugar juice is carried out. Heating temperature is 65 to 105° C., and preferably 80 to 105° C. When the heating temperature is lower than 65° C., sucrose degrading enzymes cannot be inactivated. Here, several seconds to 10 minutes of heating time is sufficient for inactivating sucrose degrading enzymes. In addition, when the heating temperature is lower than 65° C., sterilization of the sugar juice is insufficient. In order to sufficiently carry out sterilization of the sugar juice, it is preferable to adjust the heating temperature to 100° C. or higher.

Optimum conditions of the heating in a clarified step differ depending on the scale of implementation and the like. In an actual production process, it is preferable to carry out static precipitation separation for several hours after heating for precipitating suspended solids and impurities in the sugar juice. The settling time for precipitating the suspended solids and the impurities in the sugar juice is 2 to 4 hours, and preferably about 3 hours. When the settling time is less than 2 hours, it is difficult to precipitate the suspended solids and the impurities in the sugar juice.

Clarifying the sugar juice refers to removal of solid components other than sucrose contained in the sugar juice. The solid components other than sucrose include insoluble solid components such as cellulose, hemicellulose, protein, pectin and the like; and soluble solid components such as protein, pectin, amino acid, organic acid, invert sugar, ash and the like.

Removal of the solid components other than sucrose is carried out, for example, in the following way. First, lime is added to the heated sugar juice, to aggregate protein, pectin and the like. If needed, calcium hydroxide or calcium oxide is added thereto, or carbon dioxide gas is blown thereto to produce calcium carbonate, by which an aggregate of the non-saccharide components is adsorbed to calcium carbonate and precipitated. Next, insoluble components containing the aggregate and the precipitate are separated by filtration, to obtain a clarified sugar juice. The clear sugar juice mainly contains sucrose, glucose, fructose and the like.

The clear sugar juice is a clarified sugar juice, and is an aqueous solution having a sucrose concentration of not less than 9% by weight, preferably 9 to 18% by weight, more preferably 12 to 15% by weight. If the sucrose concentration is less than 9% by weight, the sucrose concentration of a concentrated solution obtained by a concentration apparatus in conventional sugar producing steps, for example, a fivefold effect evaporator tube is less than 50% by weight, melting of sugar crystal is caused in crystallizing step, recovery amount of sugar may decrease. The clear sugar juice has a purity of sucrose of not less than 50% by weight.

The clear sugar juice is then concentrated. Concentration is carried out mainly by evaporating water contained in the clear sugar juice. By means of the concentration, the clear sugar juice becomes syrup. Since the liquid volume of the syrup is reduced, the energy required to cool the syrup to a fermentation temperature is reduced as compared with that in a case where concentration is not carried out. In addition, the fermentation equipment is downsized, installation space becomes narrow, and installation cost becomes inexpensive, thereby the energy required for temperature control of the fermented solution is reduced. Furthermore, sugar concentration of the syrup is high, and fermentation efficiently progresses, thereby the production efficiency of ethanol is improved.

Brix value of the syrup is 15 to 50%, preferably 15 to 40%, and more preferably 20 to 30%. When Brix value of the syrup is less than 15%, production efficiency of ethanol is not so much improved. Brix value being more than 40% may cause fermentation failure.

When the sugar juice is squeezed juice of sugar cane, Brix value of the clear sugar juice is 10 to 20%, and typically about 13%. When the sugar juice is broth of sugar beet, Brix value of the clear sugar juice is 15 to 20%, and typically about 18%.

Volume of the concentrated sugar juice is 20 to 90% by volume, preferably 30 to 90% by volume, and more preferably 40 to 65% by volume based on the volume of the clear sugar juice. Volume of the syrup being less than 20% by volume may cause fermentation failure, and volume of the syrup being more than 90% by volume causes not so much improvement in production efficiency of ethanol.

Since the temperature of the clear sugar juice is high, it is unnecessary to carry out heating for concentration. Concentration may be carried out by, for example, introducing the clear sugar juice into an apparatus for evaporation concentration, and condensing the steam generated from the clear sugar juice into water. A specific example of the apparatus for evaporation concentration is a multiple-effect evaporator tube having connected multiple evaporator tubes which are pressure-reducible, and in which heat of steam generated in the evaporator tubes through which the liquid to be concentrated passes is recovered by a heat exchanger and sequentially utilized in the evaporator tubes through which the liquid to be concentrated passes later.

The obtained syrup is cooled, left, or heated if necessary to adjust to a temperature suitable for fermentation. The temperature suitable for fermentation is 10 to 50° C., preferably 20 to 40° C., more preferably 25 to 35° C. The clear sugar juice which is adjusted to the suitable temperature is fermented, to selectively convert the saccharide components other than sucrose in the syrup into ethanol. Such a concept of selective fermentation method is disclosed in Japanese Patent No. 4883511.

As a result of the selective fermentation, the content in the syrup of saccharide components other than sucrose becomes very small in amount. The content of invert sugar in the syrup can become substantially zero depending on conditions of the selective fermentation. The selective fermentation reduces the concentration of invert sugar in the syrup, thereby, the concentration of soluble solid components becomes low, on the other hand, the sucrose amount does not vary, and thus, the purity of sucrose is improved. The syrup after completion of the selective fermentation is not less than 70%, preferably not less than 80%, more preferably not less than 90%.

The purity of sucrose means the percentage by weight of sucrose contained in soluble solid components (Brix) in the solution.

One means for selective fermentation is fermentation carried out using a sucrose unassimilating yeast. The fermentation means the phenomenon that microorganisms such as yeast degrade saccharides under an anaerobic condition. The yeast means true fungi which normally exists in the form of single cell. The assimilation means that yeast uses as nutrition sources. Saccharides are usually degraded as being assimilated.

Yeast is a typical organism which assimilates saccharides and produces alcohol when fermenting under anaerobic conditions. Examples of sugar which the general yeast can assimilate include monosaccharides such as glucose and fructose, and disaccharides such as sucrose. In the present description, the assimilation should be interpreted as encompassing not only degradation of saccharides, but also any change which yeast can exert to saccharides such as isomerization of saccharides.

The sucrose unassimilating yeast means a yeast which assimilates saccharides other than sucrose when fermenting under anaerobic conditions, and produces alcohol. The sucrose unassimilating yeast does not exert any change to sucrose. Examples of the sucrose unassimilating yeast include a yeast having no sucrose degrading enzyme and a yeast in which all or part of the sucrose degrading enzyme genes are deleted. Invertase is known as an example of the sucrose degrading enzyme.

A microorganism having sucrose degrading enzyme genes has six sucrose degrading enzyme genes SUC 1, SUC 2, SUC 3, SUC 4, SUC 6 and SUC 7. The sucrose degrading enzyme genes are able to be destroyed by means of genetic manipulation.

Examples of the yeast having no sucrose degrading enzyme include Saccharomyces cerevisiae ATCC56805, STX347-1D, NITE BP-1587, NITE BP-1588, Saccharomyces aceti NBRC10055, Saccharomyces hienipiensis NBRC1994, Saccharomyces italicus ATCC13057, Saccharomyces dairenensis NBRC 0211, Saccharomyces transvaalensis NBRC 1625, Saccharomyces rosinii NBRC 10008, Zygosaccharomyces bisporus NBRC 1131. The yeast having no sucrose degrading enzyme is preferably a yeast having a flocculent property, and examples thereof include Saccharomyces cerevisiae NITE BP-1587, NITE BP-1588.

Another means for selective fermentation is fermentation carried out using sucrose degrading enzyme inhibitors.

Examples of the sucrose degrading enzyme inhibitors include a silver ion, a copper ion, a mercury ion, a lead ion, methyl-α-D-glucopyranoside, PCMB (p-chloromercuribenzoate), glucosyl-D-psicose and the like.

Operation and conditions for fermenting the syrup can be carried out by a method known to one skilled in the art, and include, for example, a batch method wherein fermentation is carried out by adding fermentation microorganisms and sugar juice in a given ratio, a continuous method wherein fermentation is carried out by immobilizing fermentation microorganisms and thereafter continuously feeding sugar juice, and the like.

However, in the method of the present invention, since inactivation of microorganisms and removal of foreign substances are carried out by the above-mentioned clarifying step, sucrose degradation by the microorganisms such as wild yeast, lactobacillus, acetobacter or the like is not generated upon fermentation. In addition, production of the products other than ethanol (for example, lactic acid, acetic acid or the like) from invert sugar is prevented. Therefore, ethanol fermentation can be carried out with high efficiency. In addition, the yeast obtained after the syrup is fermented does not contain microorganisms nor foreign substances since the microorganisms has been inactivated and the foreign substances has been removed during the clarifying step, and therefore, the yeast after fermentation can repeatedly be used.

The amount of the yeast added to the syrup upon fermenting the syrup is 5 g/L or more, preferably 10 to 100 g/L, and more preferably 15 to 60 g/L in wet weight. An amount of the yeast to be added of less than 5 g/L does not progress fermentation, and an excessively large amount causes inefficient separation of the liquid from the yeast upon recovery of the yeast.

The fermented solution obtained as a result of the fermentation contains the yeast, ethanol, water, sucrose, mineral, amino acid and the like. After the fermentation is completed, the yeast is separated.

The fermented solution is thereafter heated to a concentration temperature appropriate for evaporating ethanol and water. Since liquid volume of the fermented solution is reduced in the first concentration, the energy required for heating the fermented solution to a concentration temperature is reduced as compared with that in a case where concentration is not carried out.

Next, the fermented solution is concentrated again. The second concentration is carried out for recovering ethanol from the fermented solution and producing raw sugar from the fermented solution.

The recovery of ethanol from the fermented solution can be carried out by a method known to one skilled in the art, and the method is, for example, separation of ethanol by distillation. When ethanol separation by distillation is carried out, the sugar juice is concentrated at the same time. Thus, it is unnecessary to carry out heat concentration once again in production of raw sugar, and both time and energy can be saved.

In one preferred embodiment, in order to concentrate the clear sugar juice and concentrate the fermented solution, a multiple-effect evaporator tube is used. As for the multiple-effect evaporator tube, the more evaporator tubes are used, the more vapor to be used can be saved, but the concentration efficiency becomes low. Therefore, in general, a multiple-effect evaporator tube equipped with 4 to 5 evaporator tubes is used.

The clear sugar juice is let pass through an evaporator tube placed in the first position of the multiple-effect evaporator tube, and thereafter temporarily drawn in a concentrated state before introduced to an evaporator tube placed in the last position. The number of the evaporator tubes through which the clear sugar juice passes is appropriately determined so that appropriate Brix value can be provided to the syrup. Then, the syrup is cooled to a fermentation temperature, and fermentation is carried out. The obtained fermented solution is heated to a concentration temperature.

Then, the fermented solution heated to the concentration temperature is introduced to an evaporator tube placed in the next position of the evaporator tube from which the syrup is drawn. In the evaporator tube into which the fermented solution is introduced, concentration progresses, and ethanol and water are recovered.

The production of raw sugar from the fermented solution can be carried out by a method known to one skilled in the art, and the method is, for example, crystallization of sucrose, or the like. Specifically, a part of the syrup is heated under a suction reduced pressure, and the residual syrup is gradually incorporated so that the degree of supersaturation is kept within 1.1 to 1.2 to let sugar crystal grow large. A sugar crystal having a predetermined size or larger, and the concentrate is then separated into a sugar crystal and a high-viscosity with a centrifugal machine.

The high-viscosity separated from the sugar crystal is generally referred to as molasses. The molasses may be mixed with the syrup in an appropriate amount, to be used again as a fermentation raw material. Thus, the utilization efficiency of the saccharide components contained in the molasses is further improved.

### EXAMPLES

Although the present invention is explained more specifically by means of the examples described below, the present invention is not limited thereto.

### Reference Example 1

### Demonstration of the Process of Fermenting a Clear Sugar Juice in a Case where a Yeast Having No Sucrose Degrading Enzyme is Used, Using Sugar Cane as a Raw Material

### (1) Compressing Step

Three thousand and two hundred grams of stalk portions of sugar cane after harvesting were compressed with a roll mill, to obtain 3,130 g of a squeezed juice.

Here, purity of sucrose refers to % by weight of sucrose contained in soluble solid components (Brix) of the clear sugar juice.

### (2) Heating and Clarifying Step

The squeezed juice was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The aggregate suspended solids and the impurities were filtrated with a filter, to separate 3,000 g weight of a clear sugar juice (sucrose content=253 g, invert sugar content=81 g, purity of sucrose=70%). Here, microorganisms contained in the squeezed juice were sterilized by heating.

### (3) Cooling Step

The obtained clear sugar juice was cooled from 95° C. to 30° C. The energy required for the cooling was 195 kJ.

### (4) Fermentation Step

The obtained clear sugar juice was transferred to a 5 L-jar fermenter, and thereafter 150 g in wet weight of a flocculent yeast Saccharomyces cerevisiae (STX347-1D) having no sucrose degrading enzyme was inoculated thereto. Ethanol fermentation was carried out at 30° C. for 4 hours. The yeast had been previously precultured in YM media, and used. After completion of fermentation, the yeast was recovered by precipitation separation, to separate 3,100 g of a fermented solution (ethanol concentration: 1.1% by weight, sucrose content=253 g, invert sugar content=0 g).

### (5) Ethanol Distillation and Sugar Juice Concentration Step

The fermented solution was heated for temperature rise to 70° C. under reduced pressure, and 33 g of evaporated ethanol was cooled and recovered. Thereafter, water was continuously evaporated, to obtain 468 g of a syrup (sucrose content=253 g, invert sugar content=0 g, purity of sucrose=90%). The energy required for raising the temperature of the fermented solution was 124 kJ.

### (6) Crystallization Step

A half of the sugar juice was drawn out and further heated under reduced pressure, and concentrated to a degree of supersaturation of sucrose of 1.2. Thereafter, 23 g of seed crystals of sugar (particle size: 250 μm) were added thereto, and crystallization was carried out for about 3 hours, with adding the residual syrup in small portions.

### (7) Raw Sugar—Molasses Separation Step

A mixture of the crystallized sugar and molasses was centrifuged in a perforated wall type centrifugal machine using a filter fabric with 50 to 100 μm mesh at 3,000 rpm for 20 minutes, to separate 174 g of raw sugar (sucrose recovery rate=69%: excluding the amount of the added seed crystal) and 112 g of molasses.

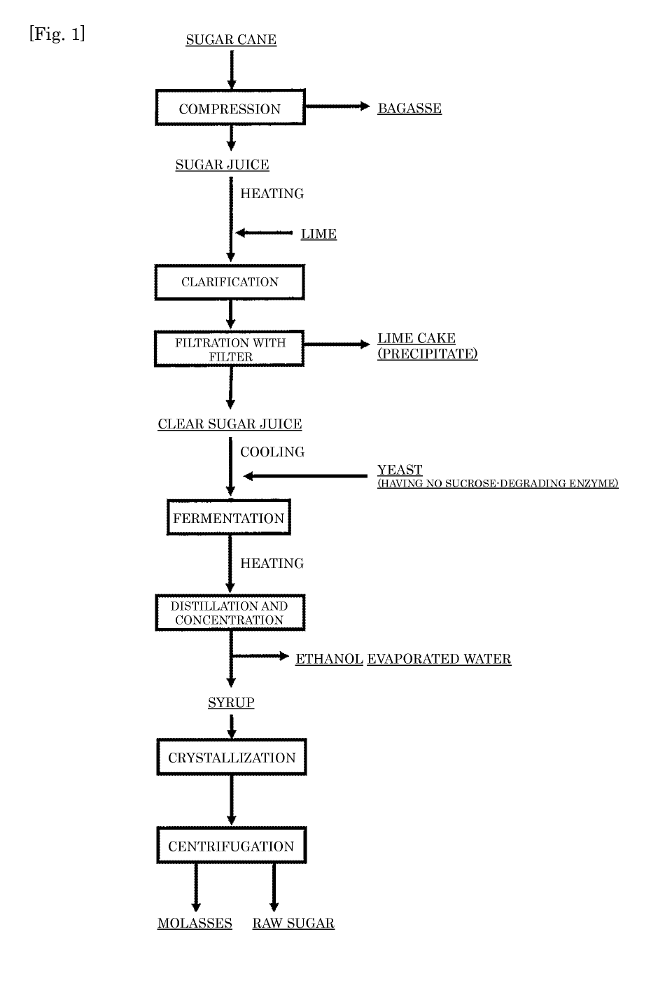

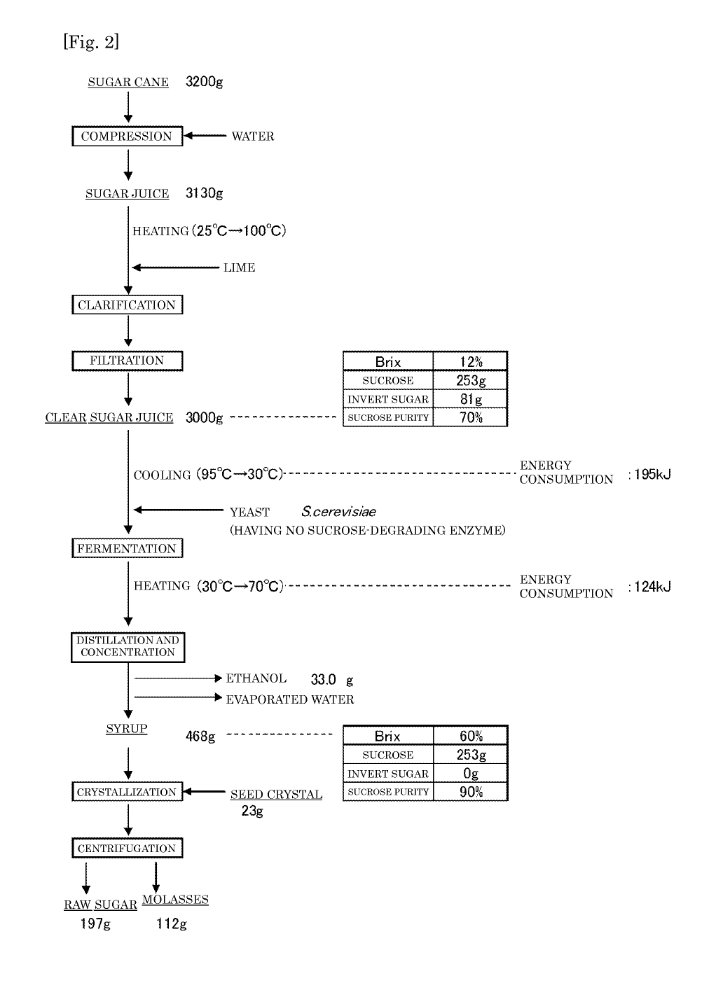

A flow diagram of the production process is shown in FIG. 1, and the result of the material balance is shown in FIG. 2.

### Comparative Example 1

### Demonstration of the Process of Fermenting a Squeezed Juice in a Case where a Yeast Having No Sucrose Degrading Enzyme is Used, Using Sugar Cane as a Raw Material

### (1) Compressing Step

Three thousand grams of stalk portions of sugar cane (NiF8) after harvesting were cut with a shredder, and thereafter compressed with a quadruple roll mill, to obtain 2,843 mL of a squeezed juice (weight of the squeezed juice=2,985 g, sucrose content=351 g, invert sugar content=112 g, purity of sucrose=63.9%).

### (2-1) Fermentation Step

The obtained squeezed juice was transferred to a 5 L-jar fermenter, and thereafter 142 g in wet weight of a flocculent yeast Saccharomyces cerevisiae (STX347-1D) having no sucrose degrading enzyme was inoculated thereto. Ethanol fermentation was carried out at 30° C. under an anaerobic condition for 24 hours. The yeast had been previously precultured in YM media, and used. After completion of fermentation, total amount of 245 g of the yeast was recovered by precipitation separation, to separate 2,822 g of a fermented solution (ethanol concentration: 2.16% by weight, sucrose content=281 g, invert sugar content=15 g).

### (2-2) Heating and Clarifying Step

The fermented solution was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The aggregated suspended solids and the impurities were filtrated with a filter, to separate 2,719 g of a clear sugar juice (ethanol concentration: 1.53% by weight, sucrose content=277 g, invert sugar content=15 g, purity of sucrose=68.6%). Unlike Example 1, 19 g of ethanol was evaporated in the heating step.

### (3) Ethanol Distillation and Sugar Juice Concentration Step

The clear sugar juice was transferred to a 5 L-evaporator tube and heated under reduced pressure, and 42 g of evaporated ethanol was cooled and recovered. Thereafter, 2,104 mL of water was continuously evaporated, to obtain 573 g of a syrup (sucrose content=277 g, invert sugar content=15 g, purity of sucrose=80.6%).

### (4) Crystallization Step

A half of the sugar juice was drawn out and further heated under reduced pressure, and concentrated to a degree of supersaturation of sucrose of 1.2. Thereafter, 29 g of seed crystals of sugar (particle size: 250 μm) were added thereto, and crystallization was carried out for about 3 hours, with adding the residual syrup in small portions.

### (5) Raw Sugar—Molasses Separation Step

A mixture of the crystallized sugar and molasses was centrifuged in a perforated wall type centrifugal machine using a filter fabric with 50 to 100 mesh at 3,000 rpm for 20 minutes, to separate 186 g of raw sugar (sucrose recovery rate=67%: excluding the amount of the added seed crystal) and 172 g of molasses (sucrose content=97 g, invert sugar content=12 g, purity of sucrose=61.3%).

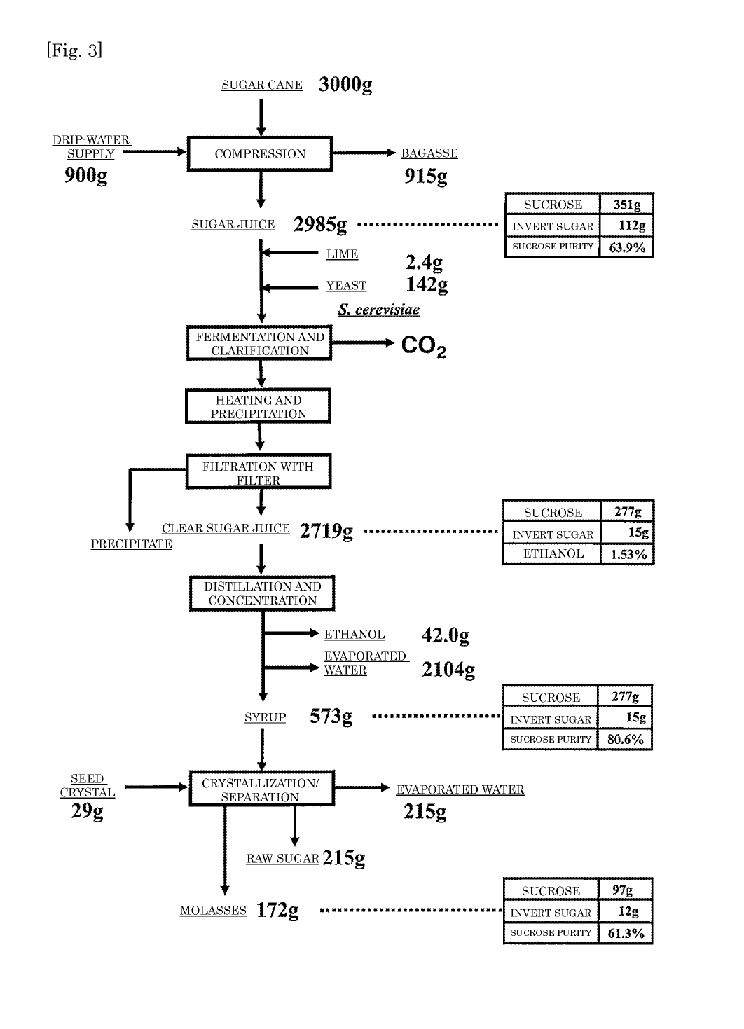

The result of the material balance of Comparative Example 1 is shown in FIG. 3.

### Example 1

### Demonstration of the Process of Fermenting a Syrup (Brix=20) in a Case where a Yeast Having No Sucrose Degrading Enzyme is Used, Using Sugar Cane as a Raw Material

### (1) Compressing Step

Three thousand and two hundred grams of stalk portions of sugar cane after harvesting were compressed with a roll mill, to obtain 3,130 g of a squeezed juice

### (2) Heating, Settling and Clarifying Step

The squeezed juice was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The squeezed juice was settled for 3 hours to precipitate the aggregated impurities. The aggregated suspended solids and the impurities were filtrated with a filter, to separate 3,000 g of a clear sugar juice (sucrose content=253 g, invert sugar content=81 g, purity of sucrose=70%). Upon filtration with a filter, since the impurities were precipitated, filtration rate was shortened. Here, in the clear sugar juice, the microorganisms contained in the squeezed juice was sterilized by heating.

### (3) Concentration Step

The clear sugar juice was heated under reduced pressure, to obtain 1,800 g of a syrup (sucrose content=253 g, invert sugar content=81 g, purity of sucrose=70%).

### (4) Cooling Step

The obtained syrup was cooled from 95° C. to 30° C. The energy required for the cooling is 117 kJ.

### (5) Fermentation Step

After the cooling of the syrup, the syrup was transferred to a 5 L-jar fermenter, and thereafter 90 g in wet weight of a flocculent yeast Saccharomyces cerevisiae (STX347-1D) having no sucrose degrading enzyme was inoculated thereto. Ethanol fermentation was carried out at 30° C. for 5 hours. The yeast had been previously precultured in YM media, and used. After completion of fermentation, the yeast was recovered by precipitation separation, to separate 1,840 g of a fermented solution (ethanol concentration: 1.9% by weight, sucrose content=253 g, invert sugar content=0 g).

### (6) Ethanol Distillation and Sugar Juice Concentration Step

The fermented solution was heated for temperature rise from 30° C. to 70° C. under reduced pressure, and 33 g of evaporated ethanol was cooled and recovered. Thereafter, water content was continuously evaporated, to obtain 464 g of a syrup (sucrose content=253 g, invert sugar content=0 g, purity of sucrose=91%). The energy required for raising the temperature of the fermented solution is 74 kJ.

### (7) Crystallization Step

A half of the sugar juice was drawn out and further heated under reduced pressure, and concentrated to a degree of supersaturation of sucrose of 1.2. Thereafter, 23 g of seed crystals of sugar (particle size: 250 μm) were added thereto, and crystallization was carried out for about 3 hours, with adding the residual syrup in small portions.

### (8) Raw Sugar—Molasses Separation Step

A mixture of the crystallized sugar and molasses was centrifuged in a perforated wall type centrifugal machine using a filter fabric with 50 to 100 μm mesh at 3,000 rpm for 20 minutes, to separate 176 g of raw sugar (sucrose recovery rate=70%: excluding the amount of the added seed crystal) and 108 g of molasses. The raw sugar amount 176 g is the number left by subtracting the seed crystals amount 23 g from recovered raw sugar amount 199 g.

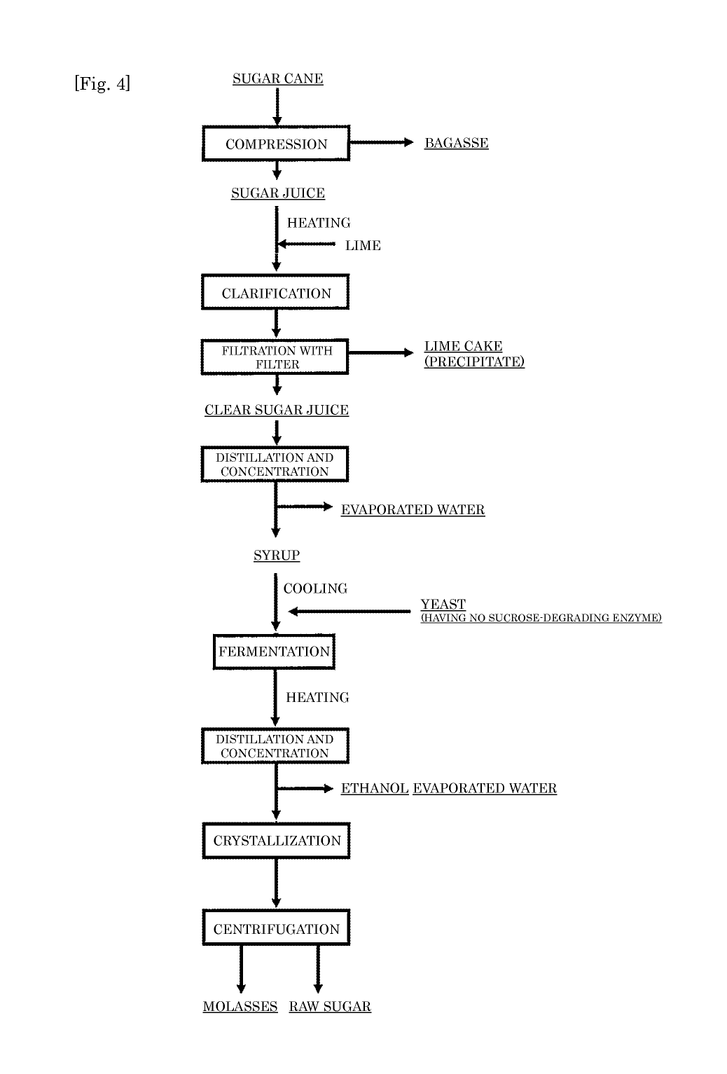

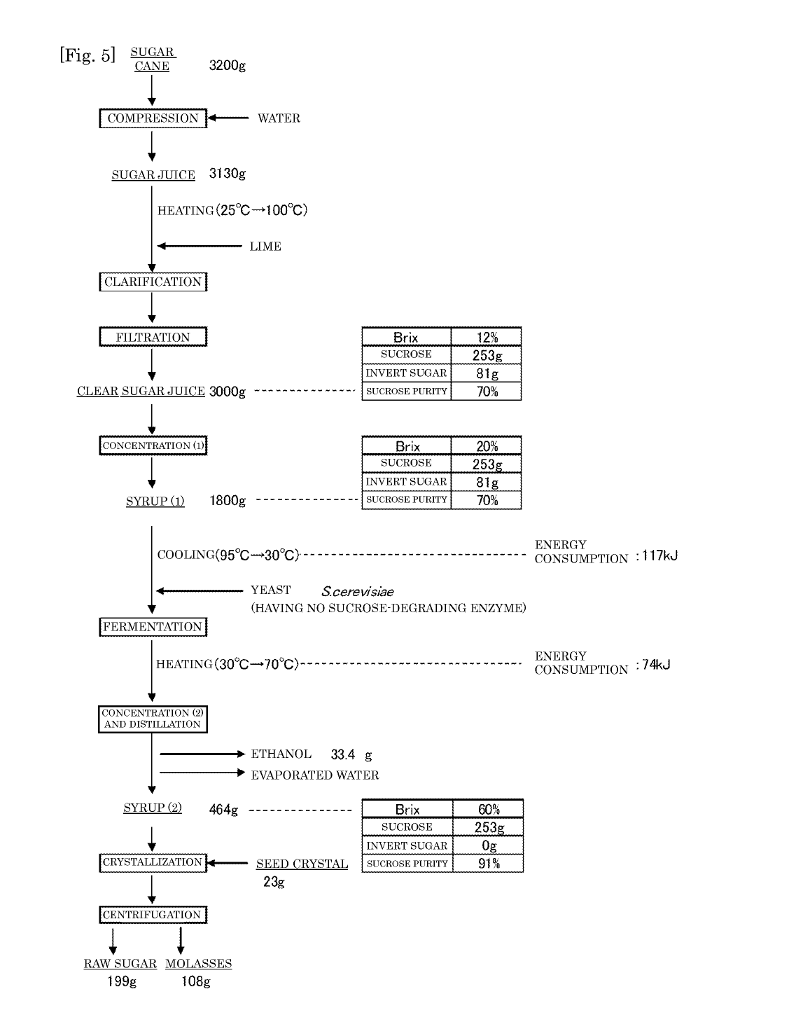

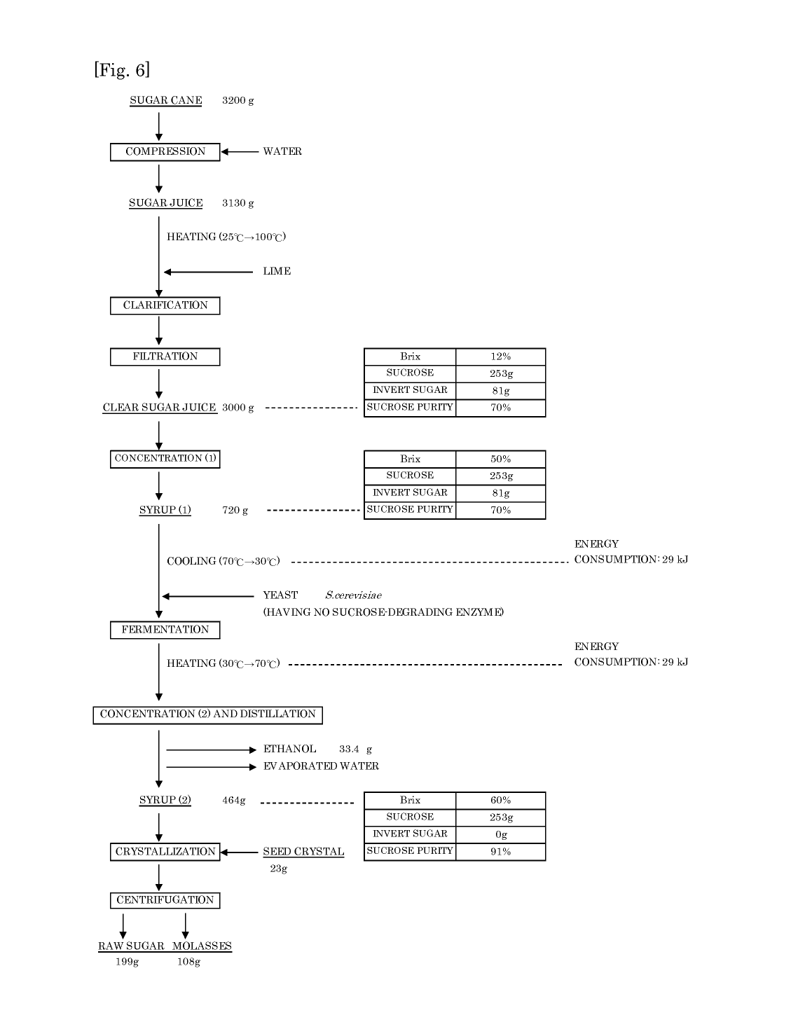

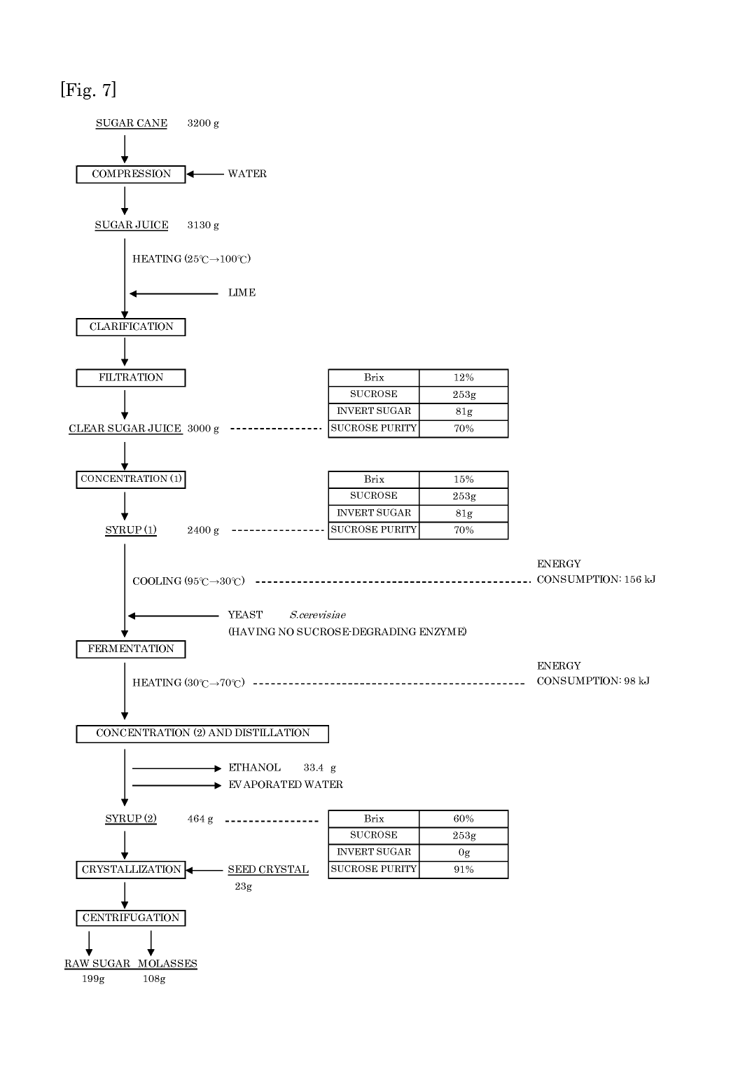

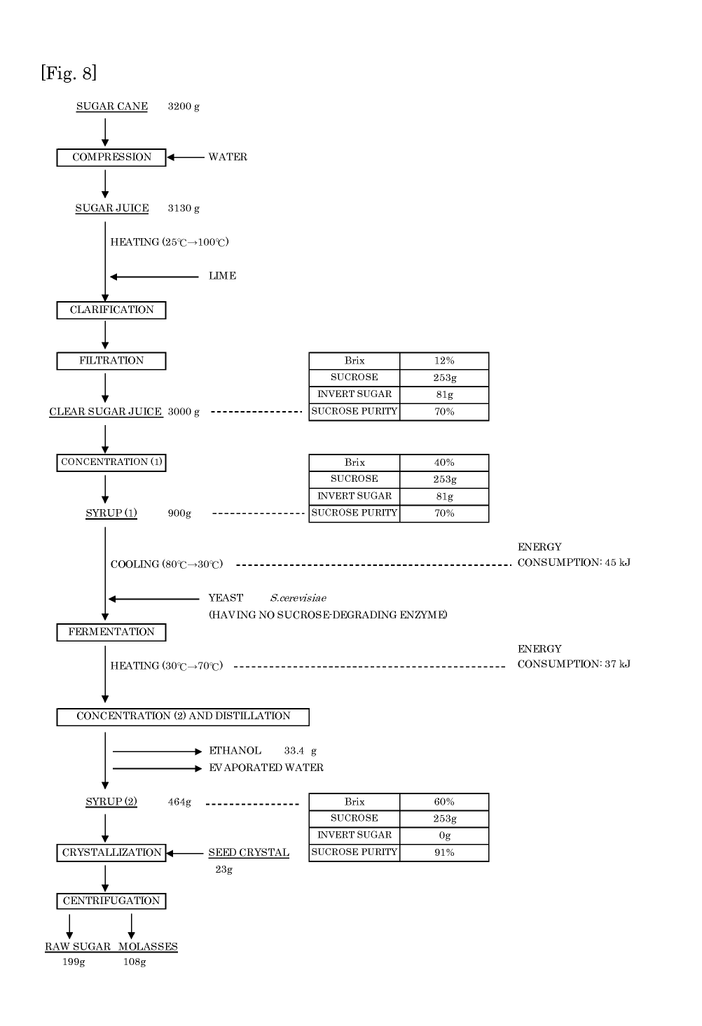

A flow diagram of the production process is shown in FIG. 4, and the result of the material balance is shown in FIG. 5. In Example 1, the amount of energy required for cooling the sugar juice to the fermentation temperature and heating the sugar juice to the concentration temperature after fermentation was 191 kJ, and the energy efficiency was substantially improved as compared with that of Reference Example 1, which required 319 kJ.

### Example 2

### (Demonstration of the Process of Fermenting a Syrup (Brix=50) in a Case where a Yeast Having No Sucrose Degrading Enzyme is Used, Using Sugar Cane as a Raw Material

### (1) Compressing Step

Three thousand and two hundred grams of stalk portions of sugar cane after harvesting were compressed with a roll mill, to obtain 3,130 g of a squeezed juice

### (2) Heating, Settling and Clarifying Step

The squeezed juice was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The squeezed juice was settled for 3 hours to precipitate the aggregated impurities. The aggregated suspended solids and the impurities were filtrated with a filter, to separate 3,000 g of a clear sugar juice (sucrose content=253 g, invert sugar content=81 g, purity of sucrose=70%). Upon filtration with a filter, since the impurities were precipitated, filtration rate was shortened. Here, in the clear sugar juice, the microorganisms contained in the squeezed juice was sterilized by heating.

### (3) Concentration Step

The clear sugar juice was heated under reduced pressure, to obtain 720 g of a syrup (sucrose content=253 g, invert sugar content=81 g, purity of sucrose=70%).

### (4) Cooling Step

The obtained syrup was cooled from 70° C. to 30° C. The energy required for the cooling is 29 kJ.

### (5) Fermentation Step

After the cooling of the syrup, the syrup was transferred to a 5 L-jar fermenter, and thereafter 36 g in wet weight of a flocculent yeast Saccharomyces cerevisiae (STX347-1D) having no sucrose degrading enzyme was inoculated thereto. Ethanol fermentation was carried out at 30° C. for 10 hours. The yeast had been previously precultured in YM media, and used. After completion of fermentation, the yeast was recovered by precipitation separation and centrifugation separation, to separate 736 g of a fermented solution (ethanol concentration: 4.8% by weight, sucrose content=253 g, invert sugar content=0 g).

### (6) Ethanol Distillation and Sugar Juice Concentration Step

The fermented solution was heated for temperature rise from 30° C. to 70° C. under reduced pressure, and 33 g of evaporated ethanol was cooled and recovered. Thereafter, water content was continuously evaporated, to obtain 464 g of a syrup (sucrose content=253 g, invert sugar content=0 g, purity of sucrose=91%). The energy required for raising the temperature of the fermented solution is 29 kJ.

### (7) Crystallization Step

A half of the sugar juice was drawn out and further heated under reduced pressure, and concentrated to a degree of supersaturation of sucrose of 1.2. Thereafter, 23 g of seed crystals of sugar (particle size: 250 μm) were added thereto, and crystallization was carried out for about 3 hours, with adding the residual syrup in small portions.

### (8) Raw Sugar—Molasses Separation Step

A mixture of the crystallized sugar and molasses was centrifuged in a perforated wall type centrifugal machine using a filter fabric with 50 to 100 mesh at 3,000 rpm for 20 minutes, to separate 176 g of raw sugar (sucrose recovery rate=70%: excluding the amount of the added seed crystal) and 108 g of molasses. The raw sugar amount 176 g is the number left by subtracting the seed crystals amount 23 g from recovered raw sugar amount 199 g.

The result of the material balance is shown in FIG. 6. In Example 2, the amount of energy required for cooling the sugar juice to the fermentation temperature and heating the sugar juice to the concentration temperature after fermentation was 58 kJ, and the energy efficiency was substantially improved as compared with that of Reference Example 1, which required 319 kJ.

### Example 3

### Demonstration of the Process of Fermenting a Syrup (Brix=15) in a Case where a Yeast Having No Sucrose Degrading Enzyme is Used, Using Sugar Cane as a Raw Material

### (1) Compressing Step

Three thousand and two hundred grams of stalk portions of sugar cane after harvesting were compressed with a roll mill, to obtain 3,130 g of a squeezed juice

### (2) Heating, Settling and Clarifying Step

The squeezed juice was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The squeezed juice was settled for 3 hours to precipitate the aggregated impurities. The aggregated suspended solids and the impurities were filtrated with a filter, to separate 3,000 g of a clear sugar juice (sucrose content=253 g, invert sugar content=81 g, purity of sucrose=70%). Upon filtration with a filter, since the impurities were precipitated, filtration rate was shortened. Here, in the clear sugar juice, the microorganisms contained in the squeezed juice was sterilized by heating.

### (3) Concentration Step

The clear sugar juice was heated under reduced pressure, to obtain 2,400 g of a syrup (sucrose content=253 g, invert sugar content=81 g, purity of sucrose=70%).

### (4) Cooling Step

The obtained syrup was cooled from 95° C. to 30° C. The energy required for the cooling is 156 kJ.

### (5) Fermentation Step

After the cooling of the syrup, the syrup was transferred to a 5 L-jar fermenter, and thereafter 120 g in wet weight of a flocculent yeast Saccharomyces cerevisiae (STX347-1D) having no sucrose degrading enzyme was inoculated thereto. Ethanol fermentation was carried out at 30° C. for 5 hours. The yeast had been previously precultured in YM media, and used. After completion of fermentation, the yeast was recovered by precipitation separation, to separate 2,450 g of a fermented solution (ethanol concentration: 1.5% by weight, sucrose content=253 g, invert sugar content=0 g).

### (6) Ethanol Distillation and Sugar Juice Concentration Step

The fermented solution was heated for temperature rise from 30° C. to 70° C. under reduced pressure, and 33 g of evaporated ethanol was cooled and recovered. Thereafter, water content was continuously evaporated, to obtain 464 g of a syrup (sucrose content=253 g, invert sugar content=0 g, purity of sucrose=91%). The energy required for raising the temperature of the fermented solution is 98 kJ.

### (7) Crystallization Step

A half of the sugar juice was drawn out and further heated under reduced pressure, and concentrated to a degree of supersaturation of sucrose of 1.2. Thereafter, 23 g of seed crystals of sugar (particle size: 250 μm) were added thereto, and crystallization was carried out for about 3 hours, with adding the residual syrup in small portions.

### (8) Raw Sugar—Molasses Separation Step

A mixture of the crystallized sugar and molasses was centrifuged in a perforated wall type centrifugal machine using a filter fabric with 50 to 100 mesh at 3,000 rpm for 20 minutes, to separate 176 g of raw sugar (sucrose recovery rate=70%: excluding the amount of the added seed crystal) and 108 g of molasses. The raw sugar amount 176 g is the number left by subtracting the seed crystals amount 23 g from recovered raw sugar amount 199 g.

The result of the material balance is shown in FIG. 7. In Example 3, the amount of energy required for cooling the sugar juice to the fermentation temperature and heating the sugar juice to the concentration temperature after fermentation was 254 kJ, and the energy efficiency was substantially improved as compared with that of Reference Example 1, which required 319 kJ.

### Example 4

### Demonstration of the Process of Fermenting a Syrup (Brix=40) in a Case where a Flocculent Yeast Having No Sucrose Degrading Enzyme is Used, Using Sugar Cane as a Raw Material

### (1) Compressing Step

Three thousand and two hundred grams of stalk portions of sugar cane after harvesting were compressed with a roll mill, to obtain 3,130 g of a squeezed juice

### (2) Heating, Settling and Clarifying Step

The squeezed juice was transferred to a 5 L-beaker, and heated at 100° C. for 10 minutes. Next, 0.085% by weight of slaked lime Ca(OH)₂ based on the weight of the squeezed juice was added thereto, to adjust pH and aggregate suspended solids and impurities. The squeezed juice was settled for 3 hours to precipitate the aggregated impurities. The aggregated suspended solids and the impurities were filtrated with a filter, to separate 3,000 g of a clear sugar juice (sucrose content=253 g, invert sugar content=81 g, purity of sucrose=70%). Upon filtration with a filter, since the impurities were precipitated, filtration rate was shortened. Here, in the clear sugar juice, the microorganisms contained in the squeezed juice was sterilized by heating.

### (3) Concentration Step

The clear sugar juice was heated under reduced pressure, to obtain 900 g of a syrup (sucrose content=253 g, invert sugar content=81 g, purity of sucrose=70%).

### (4) Cooling Step

The obtained syrup was cooled from 95° C. to 30° C. The energy required for the cooling is 45 kJ.

### (5) Fermentation Step

After the cooling of the syrup, the syrup was transferred to a 5 L-jar fermenter, and thereafter 45 g in wet weight of a flocculent yeast Saccharomyces cerevisiae (NITE BP-1587) having no sucrose degrading enzyme was inoculated thereto. Ethanol fermentation was carried out at 30° C. for 5 hours. The yeast had been previously precultured in YM media, and used. After completion of fermentation, the yeast was recovered by precipitation separation, to separate 920 g of a fermented solution (ethanol concentration: 3.8% by weight, sucrose content=253 g, invert sugar content=0 g).

### (6) Ethanol Distillation and Sugar Juice Concentration Step

The fermented solution was heated for temperature rise from 30° C. to 70° C. under reduced pressure, and 33 g of evaporated ethanol was cooled and recovered. Thereafter, water content was continuously evaporated, to obtain 464 g of a syrup (sucrose content=253 g, invert sugar content=0 g, purity of sucrose=91%). The energy required for raising the temperature of the fermented solution is 37 kJ.

### (7) Crystallization Step

A half of the sugar juice was drawn out and further heated under reduced pressure, and concentrated to a degree of supersaturation of sucrose of 1.2. Thereafter, 23 g of seed crystals of sugar (particle size: 250 μm) were added thereto, and crystallization was carried out for about 3 hours, with adding the residual syrup in small portions.

### (8) Raw Sugar—Molasses Separation Step

A mixture of the crystallized sugar and molasses was centrifuged in a perforated wall type centrifugal machine using a filter fabric with 50 to 100 mesh at 3,000 rpm for 20 minutes, to separate 176 g of raw sugar (sucrose recovery rate=70%: excluding the amount of the added seed crystal) and 108 g of molasses. The raw sugar amount 176 g is the number left by subtracting the seed crystals amount 23 g from recovered raw sugar amount 199 g.

The result of the material balance is shown in FIG. 8. In Example 4, the amount of energy required for cooling the sugar juice to the fermentation temperature and heating the sugar juice to the concentration temperature after fermentation was 82 kJ, and the energy efficiency was substantially improved as compared with that of Reference Example 1, which required 319 kJ.

1-7. (canceled)

8. A method for producing raw sugar and ethanol comprising the steps of:
heating and clarifying a plant-origin sugar juice;
concentrating the clear sugar juice so that Brix value of the clear sugar juice is 15 to 40%;
cooling the syrup to a fermentation temperature;
fermenting the syrup, thereby selectively converting the saccharide components other than sucrose in the syrup into ethanol;
concentrating the fermented solution; and
crystallizing sugar from the concentrated fermented solution.

9. A method for producing raw sugar and ethanol comprising the steps of:
heating and clarifying a plant-origin sugar juice;
introducing the clear sugar juice into a multiple-effect evaporator tube;
concentrating the clear sugar juice so that Brix value of the clear sugar juice is 15 to 40% by letting the clear sugar juice pass through an evaporator tube placed in the first position of the multiple-effect evaporator tube and thereafter drawing the clear sugar juice before introducing the clear sugar juice into an evaporator tube placed in the last position of the multiple-effect evaporator tube;
cooling the syrup to a fermentation temperature;
fermenting the syrup, thereby selectively converting the saccharide components other than sucrose in the syrup into ethanol;
heating the fermented solution to a concentration temperature;
concentrating the fermented solution by letting the fermented solution pass through an evaporator tube placed in the next position of the evaporator tube from which the syrup is drawn; and
crystallizing sugar from the concentrated fermented solution.

10. The method for producing raw sugar and ethanol according to claim 8, wherein the fermentation is carried out using a sucrose unassimilating yeast.

11. The method for producing raw sugar and ethanol according to claim 8, wherein the fermentation is carried out using a yeast having no sucrose degrading enzyme.

12. The method for producing raw sugar and ethanol according to claim 8, wherein the fermentation is carried out in the presence of a sucrose degrading enzyme inhibitor.

13. The method for producing raw sugar and ethanol according to claim 8, wherein the plant is at least one kind selected from the group consisting of sugar cane, sugar beet, sugar palm, sugar maple and sorghum.

14. The method for producing raw sugar and ethanol according to claim 9, wherein the fermentation is carried out using a sucrose unassimilating yeast.

15. The method for producing raw sugar and ethanol according to claim 9, wherein the fermentation is carried out using a yeast having no sucrose degrading enzyme.

16. The method for producing raw sugar and ethanol according to claim 9, wherein the fermentation is carried out in the presence of a sucrose degrading enzyme inhibitor.

17. The method for producing raw sugar and ethanol according to claim 9, wherein the plant is at least one kind selected from the group consisting of sugar cane, sugar beet, sugar palm, sugar maple and sorghum.
